(19) United States
(12) Patent Application Publication
(10) Pub. No.: US 2026/0301342 A1
(43) Pub. Date: Oct. 1, 2026

(54) ANEURYSM MODELING, RISK PREDICTION, AND VISUALIZATION

(71) Applicant: UNIVERSITY OF PITTSBURGH - OF THE COMMONWEALTH SYSTEM OF HIGHER EDUCATION, Pittsburgh, PA (US)

(72) Inventors: Timothy K. Chung, Pittsburgh, PA (US); David A. Vorp, Pittsburgh, PA (US)

(21) Appl. No.: 19/093,735

(22) Filed: Mar. 28, 2025

(51) Int. Cl. G06T 19/20; A61B 5/00; A61B 5/01; A61B 5/02; A61B 5/0205; A61B 5/055; A61B 6/00; A61B 6/03; A61B 6/46; A61B 6/50; G06T 7/00; G06T 11/20; G06T 13/20; G06T 17/20; G06V 10/25; G06V 10/764; G16H 40/67; G16H 50/30

(52) U.S. Cl. CPC G06T 19/20; A61B 5/015; A61B 5/02014; A61B 5/02055; A61B 5/055; A61B 5/7264; A61B 5/7275; A61B 5/7425; A61B 5/7465; A61B 6/032; A61B 6/463; A61B 6/503; A61B 6/5247; A61B 6/563; G06T 7/0012; G06T 11/26; G06T 13/20; G06T 17/20; G06V 10/25; G06V 10/764; G16H 40/67; G16H 50/30; G06T 2200/04; G06T 2200/24; G06T 2207/10081; G06T 2207/10088; G06T 2207/20036; G06T 2207/30101; G06T 2210/41; G06T 2219/2012; G06V 2201/03

(57) ABSTRACT

Aspects include methods, devices, and techniques for modeling abdominal aortic aneurysms (or other vascular diseases), predicting one or more outcomes related to the aneurysm in a patient, and visualizing the modeled abdominal aortic aneurysms and/or the predicted one or more outcomes related to the aneurysm in the patient.

### CROSS-REFERENCE TO RELATED APPLICATIONS

This application claims the benefit of U.S. Patent Application Ser. No. 63/571,550, filed Mar. 29, 2024. The disclosure of the prior application is considered part of, and is incorporated by reference in, the disclosure of this application.

### STATEMENT AS TO FEDERALLY SPONSORED RESEARCH

This invention was made with government support under grant numbers TR001857 and HL156246 awarded by the National Institutes of Health (NIH). The government has certain rights in the invention.

### BACKGROUND

### 1. Technical Field

This specification describes computational techniques for analyzing abdominal aortic aneurysms and similar structures, including techniques for modeling aneurysms, predicting a risk of rupture or other outcome, and visualizing the modeled aneurysms and/or predicted risks.

### 2. Background

Abdominal aortic aneurysm (AAA) is a leading cause of death in westernized countries. The adult abdominal aorta is typically 2 centimeters (cm) in diameter, and is defined as aneurysmal when the diameter grows to exceed 3 cm or 50% of its original diameter. If left untreated, AAA may continue to grow and eventually rupture, with resulting morbidity and mortality rates exceeding 85%.

Currently, vascular surgeons use a maximum diameter criterion, typically set at 5.5 centimeters for adults, for elective endovascular aneurysm repair surgery. When aneurysm exceeds the maximum diameter criteria, vascular surgeons will perform elective endovascular repair (EVAR) surgery. Aneurysms that fall within the 3 cm to 5.5 cm range, typically referred to as “small aneurysms,” are relegated to monitoring—despite an estimated 7 s-23.4% of small aneurysms nonetheless leading to rupture. According to traditional clinical practice, the aneurysm is measured and if the outer diameter exceeds 5.5 cm then the patient may be deemed eligible for elective repair surgery. If the diameter does not exceed the 5.5 cm threshold, then the patient is typically not recommended or deemed eligible for surgical repair. The physician may instead recommend that the patient undergo periodic surveillance to monitor how the size of the aneurysm changes over time (e.g., every 6 months).

A host of patient factors contribute to the prognosis of an aneurysm over time, including gender (e.g., up to five times more prevalent in men than women), age, dyslipidemia, smoking, hypertension, family history and obesity. Although there are patient factors (mentioned previously) for small aneurysms, most surgeons will not perform elective surgery for small aneurysms due to the unknown risks of elective or emergent repair. All clinically sized aortic aneurysms (greater than the maximum diameter criterion) are typically recommended to be repaired unless the patient is not eligible due to age or other comorbidities. It is also important to note that some aneurysms may outlive their condition.

The maximum diameter criterion has been applied for clinical decision making with respect to other types of aneurysms as well. For example, cerebral aneurysm (CA) is a degenerative dilation of arteries associated with a localized weakness in the vessel wall within the brain. Without treatment, CAs can rupture, an often-fatal cerebrovascular event and among the leading causes of death in the United States. Upon discovery of CA, a clinician evaluates the CA's clinical status to assess its risk of rupture against the risk of interventional repair. Current clinical guidelines suggest that the risk of rupture outweighs the risk of intervention when the maximum diameter of CA exceeds 7.0 mm. However, up to five percent of smaller-sized CAs have nonetheless been reported to rupture despite current clinical guidelines pointing toward no intervention.

### SUMMARY

This document describes systems, methods, devices, and techniques for modeling abdominal aortic aneurysms, predicting one or more outcomes related to the aneurysm in a patient, and visualizing the modeled abdominal aortic aneurysms and/or the predicted one or more outcomes related to the aneurysm in the patient.

Some embodiments described herein include a method. The method can include obtaining, by a system of one or more computers, medical image data of a person, the medical image data comprising one or more images that each depict at least a portion of a vascular disease. The method can further include generating, by the system and using the medical image data, a three-dimensional (3D) model of the vascular disease, the 3D model defining an outer wall of the vascular diseases. The method can further include analyzing, by the system, the 3D model of the vascular disease to determine respective values for at least one morphological feature of the vascular disease and at least one biomechanical feature of the vascular disease. The method can further include using the respective values for the at least one morphological feature of the vascular disease and the at least one biomechanical feature of the vascular disease to predict one or more outcomes related to the vascular disease. The method can further include outputting a visualization of a risk assessment for the vascular disease based on the predicted one or more outcomes related to the vascular disease, wherein the visualization is configured to be rendered in at least one of augmented reality (AR) or virtual reality (VR).

Embodiments described herein can include one or more optional features. For example, the visualization can include a 3D rendering of the 3D model of the vascular disease overlayed with a heat map corresponding to the at least one morphological feature. The visualization can include a graph presenting a comparison of the at least one morphological feature to the at least one morphological feature for a general population. The visualization can include a 3D rendering of the 3D model of the vascular disease overlayed with a heat map corresponding to the at least one morphological feature and a graph presenting a comparison of the at least one morphological feature to the at least one morphological feature for a general population. The visualization can include an animation presenting a change of the at least one morphological feature over a period of time based on the predicted one or more outcomes related to the vascular disease. The animation can present the change over the period of time includes an animation for a change in surface geometry. The animation can present the change over the period of time includes an animation for a change in wall stresses represented by a heatmap. The method can further include generating a timeline of key events based on the predicted one or more outcomes related to the vascular disease. The visualization can include 3D visualizations for each of the key events. The method can further include calculating metrics related to the at least one morphological feature and selecting one or more of the metrics related to the at least one morphological feature to include in the visualization. An AR/VR viewer interface can present the selected one or more of the metrics on a virtual 3D object. The virtual 3D object can be a cube. The visualization can be configured to be viewed by a medical provider to assess a condition of the vascular disease. The visualization can be configured to be viewed by a patient and a medical provider as part of a telemedicine application. The medical provider and the patient can view a same instance of the visualization on separate AR/VR devices. The vascular disease can be an aneurysm. The aneurysm can be an abdominal aortic aneurysm. The aneurysm can be a cerebral aneurysm.

Some embodiments described herein include a system. The system can include one or more computers and one or more computer-readable media having instructions stored thereon. The instructions, when executed by the one or more computers, can cause the system to obtain medical image data, the medical image data comprising one or more images that each depict at least a portion of a vascular disease. The system can be further caused to generate, using the medical image data, a three-dimensional (3D) model of the vascular disease, the 3D model defining an outer wall of the vascular diseases. The system can be further caused to analyze the 3D model of the vascular disease to determine respective values for at least one morphological feature of the vascular disease and at least one biomechanical feature of the vascular disease. The system can be further caused to use the respective values for the at least one morphological feature of the vascular disease and the at least one biomechanical feature of the vascular disease to predict one or more outcomes related to the vascular disease. The system can be further caused to output a visualization of a risk assessment for the vascular disease based on the predicted one or more outcomes related to the vascular disease, wherein the visualization is configured to be rendered in at least one of augmented reality (AR) or virtual reality (VR).

Embodiments described herein can include one or more optional features. For example, the medical image data can be processed using a classifier to identify a segment of the medical image data that includes a region of interest. The vascular disease can be an aneurysm and the system can be further cause to generate two-dimensional (2D) graphics to compare peak wall stresses and mean wall stresses of the aneurysm to peak wall stresses and mean wall stresses of aneurysm for a population of patients. The visualization can include the 2D graphics.

The details of one or more implementations of the subject matter described in this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 illustrates an example environment for presenting a visualization for an abdominal aortic aneurysm (AAA) risk assessment.

FIG. 2 illustrates an example environment of a computing system for modeling an aneurysm from image data, generating an AAA risk prediction, and generating a visualization for an AAA risk assessment.

FIG. 3 illustrates an example method for modeling an aneurysm from image data, generating an AAA risk prediction, and generating a visualization for an AAA risk assessment.

FIG. 4 illustrates an example AAA risk assessment visualization with rendered 3D models including a heat map visualization of stresses on the surface.

FIG. 5 illustrates an example AAA risk assessment visualization with a chart presenting a comparison of a patient's condition versus a general population.

FIG. 6 illustrates an example process that enables a user to view AAA risk assessment visualizations.

FIG. 7 is a schematic flow diagram illustrating an example process for training models to make wall stress predictions.

FIG. 8 illustrates example variables for an example model used to predict stresses for an abdominal aortic aneurysm.

FIG. 9 illustrates example inputs and an output prediction for training a classifier to segment medical data.

FIG. 10 illustrates an example process for predicting wall stresses.

FIG. 11 illustrates wall stress heatmap using computational simulations (A), predicted wall stresses based on the ML prediction model (B), and percent errors at each node of the stress differences (C).

FIG. 12 illustrates an example process for implementing the artificial intelligence pipeline with continuous and incremental learning.

FIG. 13 illustrates example stress prediction results using linear regression and different machine learning models.

FIG. 14 is a block diagram of an example computer system that can be programmed and configured to carry out any of the computational techniques disclosed in this specification.

Like reference numbers and designations in the various drawings indicate like elements.

### DETAILED DESCRIPTION

This specification describes systems, methods, devices, and techniques for analyzing abdominal aortic aneurysms (AAA) and similar vascular structures, and more particularly describes techniques for modeling aneurysms and predicting a risk of aortic rupture or other outcome related to an aneurysm in a patient and generating visualizations thereof.

In many cases, a patient's understanding, accuracy, trust, anxiety, and overall satisfaction of their hospital visit is improved when a three-dimensional (3D) medical image is shown, in comparison to when a two-dimensional (2D) image is shown or when no image is shown (verbal diagnosis given). A disconnect can happen between the clinician and the patient due to the lack of intuitive data being presented, leaving the patient in a state of confusion due to the lack of intuitive data being presented. To improve clinician and patient understanding, an artificial intelligence based workflow is disclosed that provides visualizations of the biomechanical status of an organ, including outputs which can be used in augmented reality (AR) or virtual reality (VR) to present additional information to clinicians and patients.

In some examples, trained machine learning models are used. The output from these models is used to render a 3D stress map which can be viewed in 3D on an AR/VR device. In some examples, a clinician views the 3D model with a stress map to assess a patient's aneurysm.

While many of the examples herein are described in the context of AAA, the approaches can also be used to compute stresses (or other conditions) in models for a wide range of organs. For example, the techniques can be implemented for any type of systems within the body (cardiovascular, organ, musculoskeletal, etc.).

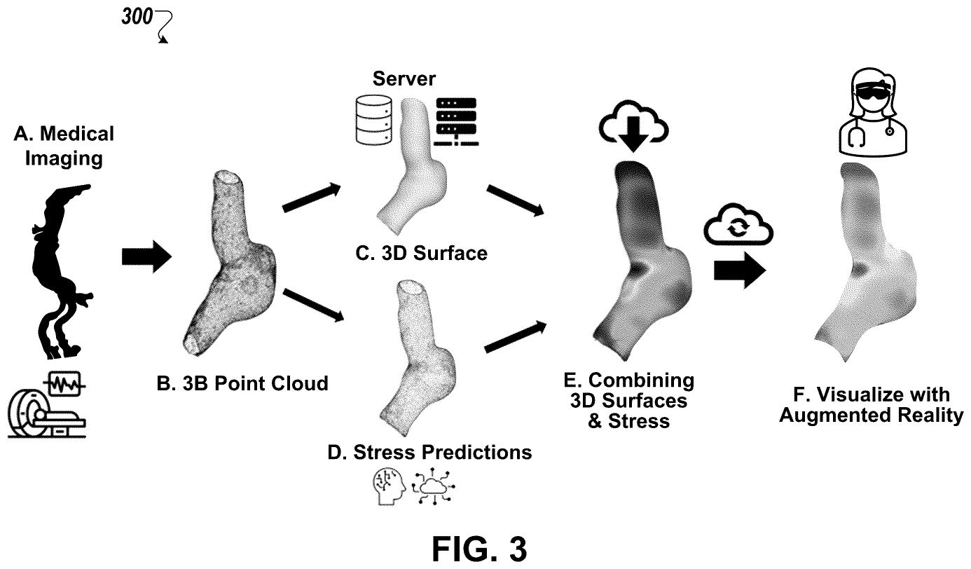

Some examples include an AI pipeline that first begins with automated image segmentation, point cloud generation, 3D surface reconstruction, and prediction of wall stresses. The disclosed AI pipeline (e.g., as shown in FIG. 3) reduces/eliminates the role of a specialized user to supplement and enhance the interaction between the clinician and patient.

Image segmentation processes disclosed herein, can include techniques to avoid poor segmentation, which would lead to an invalid 3D surface reconstruction and improper stress prediction (i.e., reduction in overall accuracy, sensitivity, and specificity of the stress prediction models). Some examples include an external control system that ensures that the region of interest closely matches the original 3D CT volume. The external control system could query the automated image segmentation results with manual or semi-automated segmentation methods. Lastly, the stress predictions can be compared to a database of aneurysms peak wall stress (PWS) and/or mean wall stress to potentially reject an outlier or more closely scrutinize the results of the AI pipeline.

Some examples include an AR system that allows a clinician to quickly assess the biomechanical wall stresses of cardiovascular diseases in medical images. The multimodal AR system can include training AI models from medical images and stress analysis submodule, a computational workstation-local server submodule, and an AR application module. The modules operate together to view medical images, visualize wall stress on aneurysms, and allow gesture-based interaction between the clinician (and/or the patient) and the generated object. Additionally, the end-user can interact with a data stream that compares the patient's aneurysm with the general population. The proposed data stream can quantify and visualize morphological measurements, wall stress metrics (peak and mean values) and compare their values to an aneurysm database.

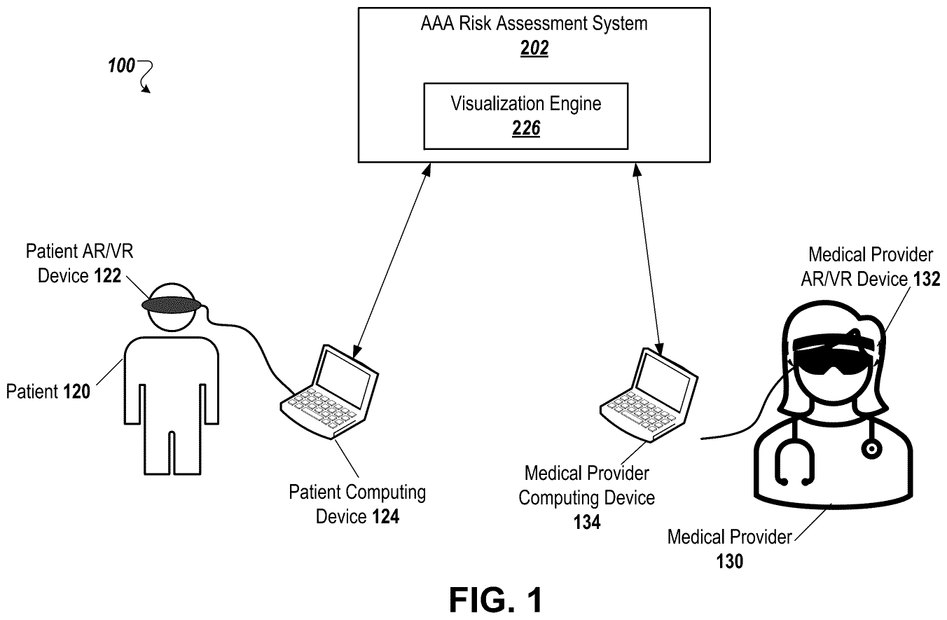

FIG. 1 illustrates an example environment 100 for presenting AAA risk assessment visualizations. The example environment 100 includes an AAA risk assessment system 202, in digital communication with a patient computing device 124 and a medical provider computing device 134. The patient 120 and the medical provider 130 view the AAA risk prediction visualizations on the patient AR/VR device 122 and the medical provider AR/VR device 132, respectively.

The patient computing device 124 and the medical provider computing device 134 are user computing devices that can operate an AR/VR viewer application. In some examples, a single computing device is used and connected to both the patient AR/VR device 122 and the medical provider AR/VR device 132. In some examples, the patient computing device 124 and the medical provider computing device 134 are optional or not required. For example, the patient AR/VR device 122 and/or the medical provider AR/VR device 132 can include a computing device with a network connectivity that is able to communicate with the AAA risk assessment system 202. Although the example shown includes both a patient 120 and a medical provider 130, the visualizations may be provided to one of the patient 120 or medical provider 130 individually.

The patient AR/VR device 122 and/or the medical provider AR/VR device 132 include a device with a display that is capable of presenting an AR/VR environment. In some examples, the AR/VR devices include a head-mounted display, processing unit, and user interface components. The AR/VR device can include sensors (such as a camera, accelerometers, gyroscopes, etc.), controllers to allow the user to interact with the AR/VR viewer.

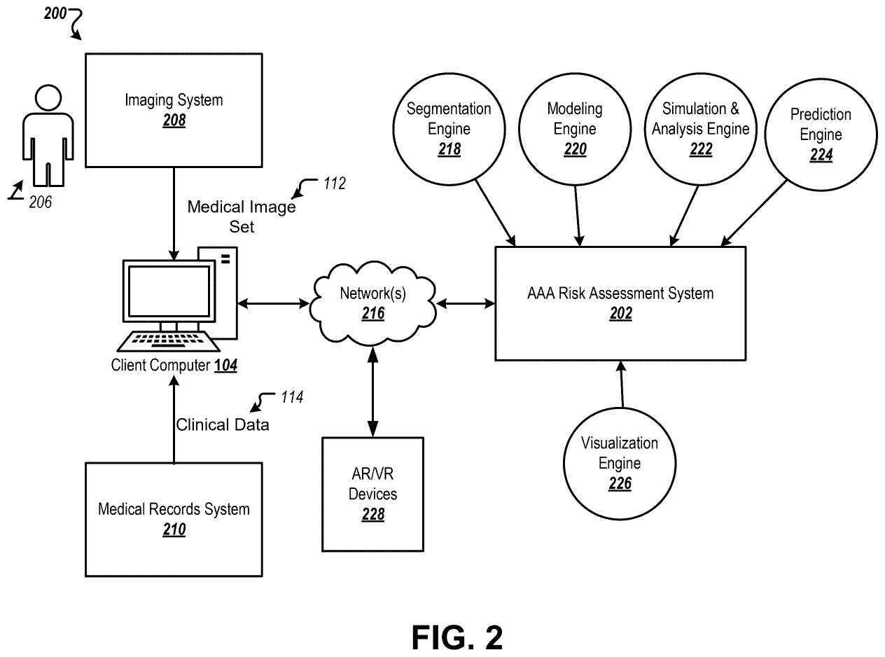

The AAA Risk Assessment system 202 can include one or more servers. In some examples, a local server that can securely store sensitive medical image data, implement some or all of the AI pipeline steps disclosed herein, perform additional quantitative analysis, and perform some or all of the 3D modeling steps for providing the AAA risk assessment visualizations to the AR/VR devices. In some examples, the AAA Risk assessment system 202 is a cloud based system. An example of the AAA risk assessment system 202 is illustrated and described in reference to FIG. 2.

As discussed above, patients understand their diagnoses better when shown a 2D or 3D image of their respective diseases. However, as discussed above, medical providers do not traditionally show patients their images as it requires additional time and processing. In some implementations, AR or VR is used to visualize abdominal aortic aneurysms AAA risk assessments as determined by the AAA risk assessment system 202. In some examples, at the AAA risk assessment system 202 medical images undergo automated segmentation, and wall stresses are predicted and mapped to the 3D surface of aneurysms to generate a 3D heat map at the visualization engine 226. In some examples, the pipeline includes introducing elements into an AR and/or VR ecosystem to view models and additional analytics, enabling medical providers and patients to view the biomechanical status without the need for a computational or imaging expert. In some implementations, a local server processes medical imaging data, generates point clouds, predicts wall stresses on individual points, and creates a 3D model with a colormap to view in AR and/or VR. In some examples, the visualization of wall stresses of cardiovascular diseases using an AR environment provides a prognostic tool to aid in clinical decision-making.

The visualization engine 226 is configured to generate AAA risk assessment visualizations. In some examples, these visualizations include animations that are overlaid on a 3D object rendering the aneurysm. For example, an animation may show changes to stress (e.g., via an animated heat map) and/or changes in surface geometry. In some examples, these changes are backward looking using historical medical imaging data for the patient. In other examples, these changes are predicted. For example using the AI pipeline disclosed herein. In some examples, a timeline/chart of key events is generated, with animations highlighting changes at each key event.

In some examples, the visualization engine 226 selects the most relevant metrics for a patient and/or practitioner. In some examples, AI is used to determine the most relevant metrics using different feature importance algorithms. In some examples, the risk assessment visualizations include an interface for toggling between metrics. In some examples, this includes a virtual cube, where each face of the cube displays a different metric and/or visualization. In some examples, as the cube is moved to expose a different face a visualization of the 3D rendering of the organ is updated.

In some examples, a 3D rendering of the organ is presented adjacent to population level analytics. In some examples, the patient data is visualized as a comparison to the population level analytics.

In some examples, the patient 120 and the medical provider 130 view a common AR/VR environment. In some examples, the patient 120 and medical provider 130 are remote to each other (e.g., located at separate geographic locations) and the common AR/VR environment is used alongside a telemedicine platform. In some of these examples, the patient 120 and the medical provider 130 view an instance (e.g., the same view and/or the same virtual environment) of virtual objects/other visualizations in AR. In some examples, the AR/VR visualizations are -provided alongside a telemedicine applications e.g., via a computer application with video or voice conferencing. In some examples, the patient views the visualization on a computing device as part of the telemedicine application (e.g., presenting the visualization on the computing device 124 without the use of the AR/VR visualization device 122.

FIG. 2 illustrates an environment 200 of a computing system for modeling an aneurysm from image data, generating an AAA risk prediction, and generating a visualization for an AAA risk assessment. In general, the environment 200 encompasses one or more computers and associated equipment (e.g., imaging system 208) in one or more locations, and each of the individual systems 202, 204, 208, 210, 218, 220, 222, 224 can also be implemented on one or more computers in one or more locations. In some contexts, environment 200 provides for interaction between a local client computer 204 and a remote service (e.g., cloud-based service) executed on the AAA risk assessment system 202 in coordination with engines 218-224. For example, local entities such as physicians, hospitals, or clinics may subscribe or pay per-use fees to a provider of the AAA risk assessment service. The client computer 204 can send requests to the AAA risk assessment system 202 via one or more networks 216 (e.g., the Internet, local area networks (LANs), wireless area networks (WANs)), and the AAA risk assessment system 202, upon receiving all necessary inputs and data for analysis, may return reports to the client computer 204 containing AAA risk prediction(s) and/or modeling results to the client computer 204. A physician may review the reports to inform recommendations and treatment plans in relation to a patient's AAA.

The on-site environment can include an imaging system 208, client computer 204, and medical records system 210. Imaging system 208 is configured to obtain a set of medical images of a patient 206, and in particular, can be adapted to capture images that depict internal organs of the patient 206 including the aorta. Imaging system 208 can emit electromagnetic radiation toward an abdominal region of the patient 206 and record signal(s) detected in response to the radiation (e.g., reflected radiation) to produce an image of the targeted area. In some implementations, imaging system 208 is a computed tomography (CT) system that uses specialized X-ray equipment to produce a series of cross-sectional images of the patient's abdominal region. Each cross-sectional image represents a slice of the targeted region, for example. In other implementations, imaging system 208 is a magnetic resonance imaging (MRI) system in which the patient 206 is positioned in a magnetic field and the system directs radio waves toward the targeted region of the patient's body to obtain images of internal structures of the body.

Medical records system 210 is configured to maintain medical records for one or more patients, and can include one or more database(s) to store medical records in a structured format that facilitates indexing and accessing of specified records. Among other forms of medical records, the medical records system 210 can store clinical data 214 that describes information about a patient 206 apart from a medical image set 212 that is nonetheless clinically relevant to assessing the risk of an abdominal aortic aneurysm in the patient 206. For example, clinical data 214 can include indications of the patient's sex or gender, age, and whether the patient exhibits risk factors such as dyslipidemia, smoking, hypertension, obesity, family history of vascular issues, diabetes, body mass index, body surface area, height, weight, coronary heart disease, history of congenital heart failure, history of COPD, dialysis/kidney failure, sepsis, SIRS, septic shock, WBC count, hematocrit percentage, Marfan's, history of myocardial infarction (heart attack), angina, swollen legs, shortness of breath, bronchitis, other respiratory diseases, limited mobility pharmaceutical use, or comorbidities.

Client computer 204 receives the set of medical images 212 acquired by imaging system 208 and clinical data 214 from medical records system 210, and generates a request for an AAA risk prediction for the patient 206. The request can be transmitted from client computer 204 to AAA risk assessment system 202 in one or more messages over networks 216, and can include all or a portion of medical image set 212 and clinical data 214 in format(s) suitable for processing by AAA risk assessment system 202.

In response to receiving a request from client computer 204, AAA risk assessment system 202 begins processing the request to generate an AAA risk prediction. The AAA risk prediction indicates a prediction for one or more possible outcomes related to the patient's aneurysm. Typically, the predicted outcomes include aortic rupture such that the AAA risk prediction would indicate a risk or likelihood of the patient's aorta rupturing due at least in part to the aneurysm. However, additional or alternative outcomes besides rupture can also be considered such as a likelihood that the aneurysm increases in size (e.g., diameter) by at least a specified amount or a likelihood that the aneurysm achieves at least a minimum size (e.g., 5.5 cm). Significantly, at the time of evaluation by the AAA risk assessment system 202, the patient's aneurysm may still be considered “small” by traditional interpretations (e.g., <5.5 cm). The AAA risk assessment system 202 may nonetheless incorporate additional clinical indices of the patient and morphological and biomechanical indices of the aneurysm to provide a richer, more holistic, and/or reliable prediction of the risk presented by a “small” aneurysm that would otherwise be available by a maximum diameter criterion alone. In some implementations, the AAA risk prediction includes a risk score that indicates a likelihood (e.g., probability) of the one or more possible outcomes related to the aneurysm being actualized. In some implementations, the AAA risk prediction includes a classification that indicates a relative stratification of the risk presented by the aneurysm. For example, the AAA risk assessment system 202 may apply pre-defined thresholds to the risk score to classify a patient's risk as high, medium, or low. Each classification can correspond to a different clinical recommendation, e.g., a high risk prediction can correspond to a recommendation for elective endovascular repair of the aneurysm, a medium risk prediction can correspond to a recommendation for more frequent surveillance/monitoring of the aneurysm (e.g., schedule the patient for follow-up appointments every 3 months), and a low risk prediction can correspond to a recommendation for less frequent surveillance/monitoring of the aneurysm (e.g., schedule the patient for follow-up appointments every 6-12 months).

AAA risk assessment system 202 employs multiple processing engines 218, 220, 222, 224, and 226 to process the request data and generate an AAA risk prediction. Although depicted in FIG. 2 as distinct modules, all or some of the engines 218, 220, 222, 224, and 226 may be merged or further split into separate processing phases according to the demands of a particular application, and moreover all or some of the engines 218, 220, 222, 224, and 226 can be implemented within or apart from the AAA risk assessment system 202. In general, a processing “engine” in this specification refers to hardware, software, or both (including firmware) configured to perform one or more functions on one or more data processing apparatus. Collectively, the AAA risk assessment system 202 in cooperation with engines 218, 220, 222, 224, and 226 provide a fully automated pipeline for analyzing a medical image set 212 and clinical data 214 to generate a computational 3D model of an abdominal aortic aneurysm, and to determines values for mechanical features of the aneurysm from the 3D model for use in generating an AAA risk prediction. Segmentation engine 218 processes all or some of the images in the medical image set 212 to isolate and extract the relevant portions of the images that depict the aorta in an aneurysmal region. Modeling engine 220 processes the segments of the medical image set 212 extracted by segmentation engine 218 to build the 3D model of the aneurysm.

With the 3D model constructed, simulation and analysis engine 222 performs finite element analysis to simulate blood flow through the aneurysm, thereby facilitating computation of stresses on the walls and other geometries of the aneurysm that could lead to rupture or other adverse outcomes. The AAA risk assessment system 202 uses results from the finite element analysis or other simulation results from engine 222 to extract respective values for one or more biomechanical features of the patient's aneurysm. Biomechanical features of the wall, lumen, and ILT can be taken under consideration. Biomechanical features can include average wall stress or tension at one or more locations of the aneurysm, peak wall stress or tension at one or more locations of the aneurysm, average von Mises stress, average max Principal Stress, failure strength, failure tension, peak min wall tension, peak rupture potential index (RPI) tension, average max tension, average min tension, inter quartile range (IQR) Mises, IQR stress, IQR RPI, IQR RPI tension, IQR min principal stress, IQR max principal stress, IQR peak wall tension, IQR Mises stress, all aforementioned metrics based on any range of percentiles, and others.

The AAA risk assessment system 202 can also obtain measurements of one or more morphological features of the aneurysm from the segmented medical images, computational 3D model, or both. These morphological measurements, or morphological feature values, can include tortuosity of the aneurysm, characteristics of the intra-luminal thrombus (ILT) region such as a maximum, minimum, and/or average thickness of the ILT region, a volume of the aneurysm, a maximum, minimum, and/or average diameter of the outer wall and/or lumen of the aneurysm, an outer wall and/or luminal wall surface area, diameter-height ratio, height ratio, diameter-diameter ratio, bulge location, asymmetry factor tortuosity, ILT volume, isoperimetric ratio, non-fusiform index, surface area of AAA sac, neck diameter, height of aneurysm sac, max principal curvature, min principal curvature, distal neck diameter, proximal neck diameter, maximum transverse diameter, lumen asymmetry, wall asymmetry, aspect ratio (height/diameter), length of AAA sac centerline, length of neck centerline, height of neck, distance between lumen centroid and centroid of maximum diameter cross section, area-averaged Gaussian curvature, mean curvature, and/or others facets of the aneurysm's geometry

The visualization engine 226 is another example of the visualization engine 226 illustrated and described in reference to FIG. 1. In some examples, the visualizations are generated by the visualization engine 226 using the visualization of medical imaging data with the visualizing of biomechanical wall stresses on aneurysm surfaces using the outputs from engines 218, 220, 222, and 224. In some examples, similar techniques can be implemented for other cardiovascular or neurovascular diseases including cerebral aneurysms and ascending thoracic aneurysms toward improving human health and interaction between patients and clinicians.

In some examples, visualization engine 226, produces a visual representation of the 3D model of the aneurysm, the risk prediction, or both. In some implementations, the visualization is produced in a virtual, augmented, or mixed reality environment. For example, a 3D model of the aneurysm can be rendered at a first virtual location and information based on the risk prediction can be rendered at a second virtual location at least partially overlaid or to the side of the 3D model. The information rendered can include the risk prediction itself (e.g., a risk score, a risk category, a predicted time to event) and information derived from the risk prediction that may be useful in interpreting the risk prediction. For instance, the system may determine that the patient belongs to a particular demographic group based on age, sex, and/or other factors, and may provide information to the patient and clinician indicating how the patient's risk prediction statistically compares to other patients in the same or related demographic group. The 3D model may also be interactive, allowing the clinician and/or patient to rotate, pan, and zoom the aneurysm. The visualization engine 226 can further colorize and render labels over the 3D model to indicate localized wall stresses or other biomechanical indices of the aneurysm. Visualization engine 226 can produce both localized and remote views to allow the 3D model and risk predictions to be viewed both in the clinician's office and remotely for in-person or remote consultations.

The AR/VR devices 228 are computing devices for viewing and interacting with an AR/VR environment including those presenting AAA risk assessment visualizations. In some examples, the AR/VR devices process real-time data, create spatial environments, eye-tracking, and an integrated operating system (OS) to communicate with the local workstation. In some examples a viewer on the AR/VR device is interacted with using gesture-based controls. In the example shown the AR/VR devices 228 are linked to the AAA risk assessment system 202 receive AAA risk assessment visualizations. In some examples, an application on a workstation local to the AR/VR device is included with the ability to quickly load medical image sets, perform image segmentation and stress prediction, and visualize the output. The AR/VR devices allow for hand gestures and eye-tracking with the additional customized interactions. In some examples, the AR/VR devices include the patient AR/VR device 122 and the medical provider AR/VR device 132, illustrated and described in reference to FIG. 1.

Techniques for predicting wall stress and other biomechanical indices of an aneurysm are described in PCT/US2020/055511, filed Oct. 14, 2020 and published Apr. 22, 2021 as WO2021/076575, the entire contents of which are incorporated by reference in their entirety into the disclosure of this specification. Techniques for aneurysm modeling and risk prediction are described in PCT/US2022/051718, filed Dec. 2, 2022 and published Jun. 9, 2023 as WO2023/102229, the entire contents of which are incorporated by reference in their entirety into the disclosure of this specification. Techniques for forecasting shapes of tissue regions using machine learning are described in U.S. Application No. 63/451,097, filed Mar. 9, 2023, the entire contents of which are incorporated by reference in their entirety into the disclosure of this specification.

FIG. 3 illustrates an example method 300 for modeling an aneurysm from image data, generating an AAA risk prediction, and generating a visualization for an AAA risk assessment. In some examples, the method 300 is implemented in the environment 200 illustrated and described in reference to FIG. 2. The method 300 includes steps A, B, C, D, E, and F.

At step A, medical imaging data is received. A wide range of medical imaging modalities can be used including ultrasound, 2D radiographs or x-rays, and 3D methods leveraging CT or magnetic resonance imaging (MRI) to capture the medical imaging data. In some examples, The medical image data uses the DICOM header file embedded inside each individual 2D image file (that makes an entire 3D volume or stack in the axial direction). The medical images are then processed by performing automated segmentation (e.g., using a trained 2D or 3D convolutional neural network, an example being a U-NET) or manual/semi-automated image segmentation.

In some examples, the medical images are segmented to isolate a region of interest from a 3D volume from a medical image set (e.g., a CT image set. Segmentation can include manual segmentation, automated segmentation, or both. Manual segmentation includes reading and viewing a 2D slice of a single image with the 3D volume to manually trace a region of interest. This may include, for abdominal aortic aneurysms, tracing the aneurysm wall and lumen, where the lumen will have a contrast agent, making the blood flow region bright white. Automated segmentation includes converting the medical image data into 2D image data into an image classifier model to automatically segment the region of interest.

At step B, a 3D point cloud is generated from the manual/semi-automated or automated segmentation. The 3D point cloud uses pixel dimensions for X, Y, and Z directions. A scaling factor is applied using information from the DICOM header file (X and Y pixel spacing) and relative position (image patient position) for the distance between 2D axial slices. The scaling factors (pixels/millimeters) are applied to the 3D point cloud to convert into ‘real-world’ units.

In some implementations, the segmented region of interest is converted into a 3D point cloud. In some of these examples this includes recovering the pixel spacing information from the DICOM header file. The pixel spacing for x and y will provide the scaling factor to convert into real-world units. For the z-spacing, several methods can be used. The first method uses the slice thickness from the DICOM header file. A second method uses the image patient position information that provides the X, Y, and Z spatial coordinates. For example, two images that are next to each other sequentially are identified and used to find the physical distance between the z values. This second method provides the z-scaling factor to convert the ROI into real-world units. The 3D point cloud is created based on the real-world values by multiplying the x, y, and z scaling factors to the volume (based on pixels).

At step C, the 3D point cloud is converted into a 3D surface. This is achieved by computing the normal of each vertex or node and finding a neighborhood of points. A Poisson reconstruction is applied to create a 3D surface of the aneurysm wall.

In some implementations, the 3D point cloud is converted to a surface mesh. A 3D surface can be reconstructed by first computing the normal and performing Poisson surface reconstruction (determine the number of neighbors for individual points). In some examples, the 3D surface is a collection of points or nodes defined in Cartesian coordinates (X, Y, and Z), where the surface is defined by connection to nodes, forming triangle elements (this can be quadrilateral elements as well).

At step D, wall stresses for the aneurysm are predicted. In some examples, several machine learning models were trained to predict the wall stresses using a pre-processing script. The pre-processing script calculated the relative position of the vertices to the centroid of the AAA, intraluminal thrombus thickness, and principal curvatures. The processed data is input into the ML models to predict wall stresses for each vertex or node.

In some implementations, a trained machine learning model is used to generate predictions on each node. In some examples, preparing the data input into the stress prediction model is automated using a script. The inputs include the aneurysm wall coordinates (X, Y, Z), ILT thickness, nodal neighbors, principal curvatures, distance to centroid are tabulated in a data-preparation step, and combinations thereof. The prepared data from an aneurysm case is input into the trained machine learning model. The wall stresses are predicted by the inputs and applied to each node, where the wall stresses per node correspond to the heatmap colors and are saved for step E.

Step E combines 3D surfaces with the predicted wall stresses. The heatmap of stresses needs to be applied to the 3D surface. This process is performed by taking the predicted wall stresses and applying a color map to each vertex defined as a red, green, and blue color (normalized between 0 and 1). The colors based on the predicted wall stresses are then baked onto the surface using Blender, and exported as an ‘.obj’,‘.vrml’, or ‘.glb’ file for visualization. The colors can also be programmatically backed into the surface using vertex interpolation to color the faces of a 3D surface mesh.

In some implementations, a 3D model that can be visualized in augmented or virtual reality is created. In some examples, creating the 3D model includes organizing the data to have X, Y, Z coordinates with the predicted wall stresses. The element connectivity from the 3D surface can be used, where the vertex colors are unique to the wall stresses levels and the 3D model visualizes the predicted wall stress values on the nodes defining the 3D surface. In some examples, an automated script runs the application programming interface, imports the data (X, Y, Z, and predicted wall stresses), and exports a 3D model with a heat map. In some examples, the new 3D model or object is uploaded via link from a local server (or via a cloud service).

In some implementations, 2D graphics and plots are generated. An expansive database with processed aneurysm results is used to compare peak wall stress (PWS) and mean wall stress (MWS). In some examples, plots are generated to compare the PWS/MWS relative to the rolling database of aneurysm patients. In some examples, raincloud plots, histograms, or boxplots are generated. In some examples, the 2D plots can be used at step F as faces of a 3D cube or other object elements to visualize in the augmented or virtual reality system.

At step F, the 3D model file is then uploaded to the augmented or virtual reality environment for visualization. The file can be accessed via cloud or local data storage and visualized in a number of 3D viewers that both the clinician and patient can view. In some examples, AR/VR view is configured to provide advanced analytics and population statistics for an expanding computational database to aid clinicians during patient follow-up visits.

In some implementations, the 3D models are visualized using augmented or virtual reality. In some examples, the 3D model is retrieved (e.g., via a native file explorer or cloud storage). The 3D model can be opened using a native AR/VR viewer on the AR/VR device, or other 3D viewer. In some examples, the model can be interacted with using the AR/VR device and the anterior or posterior views of the aneurysm can be visualized. In some examples, a patient can also use an augmented reality headset or visualize what the clinician is doing in real-time on a separate monitor. In some examples, additional data analytics from the database of a given patient is retrieved and visualized. For example, a 2D image file containing the data analytics that has been processed for a patient can be opened and presented. In some examples, provisions for applying texture maps with differing analytics can be applied to a cube (e.g., providing 6-faces for visualizing data).

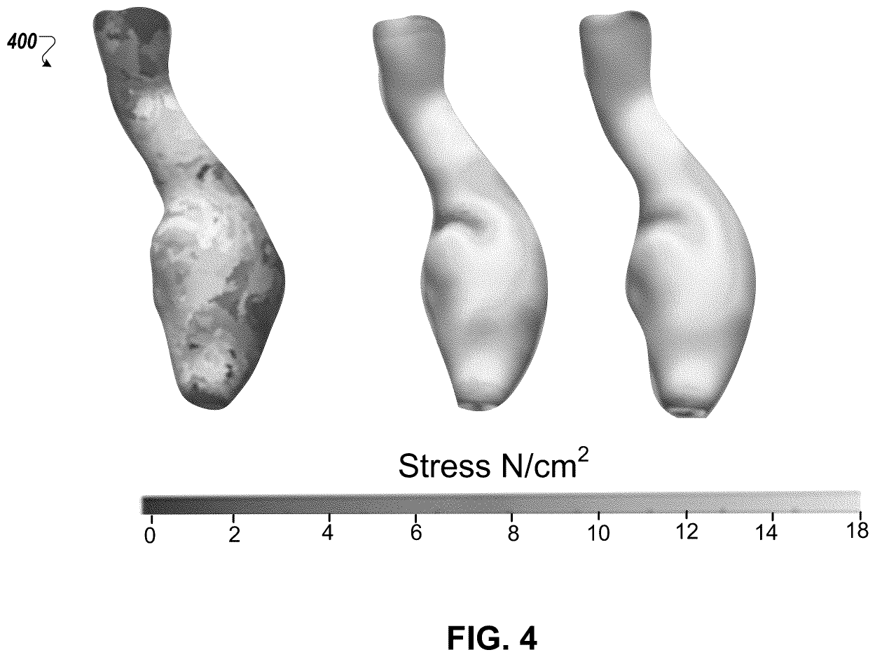

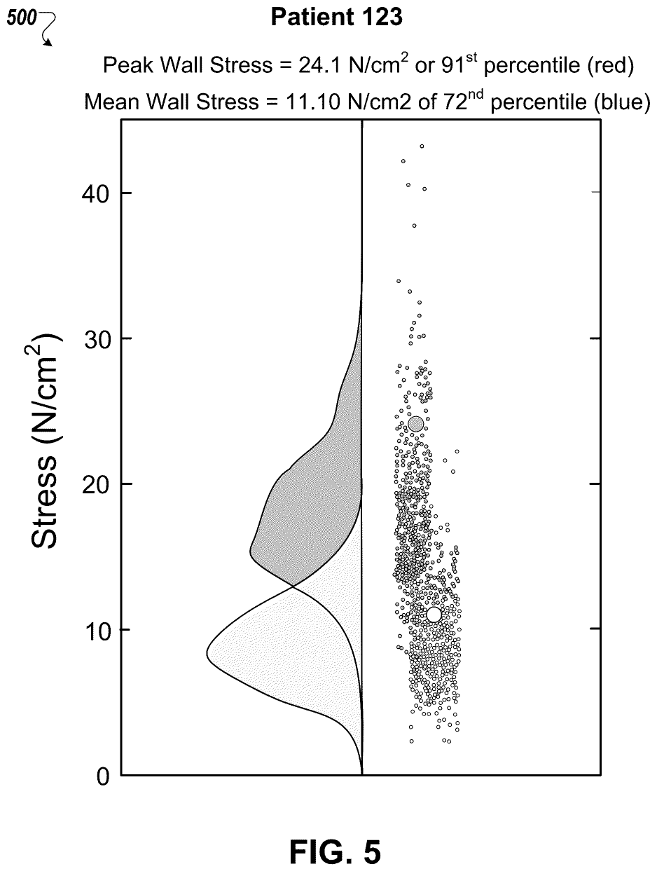

FIG. 4 illustrates an example AAA risk assessment visualization 400 with rendered 3D models including a heat map visualization on the surface. The 3D models are exported and uploaded into the augmented reality system. There are three models loaded using the file system and operating system of the hardware (e.g., Microsoft HoloLens). The three models represent the stress predictions from a fine tree model, ground truth (stress analysis), and a trilinear neural network. FIG. 5 illustrates a 2D image of the analytics used for the example 3D model with stress predictions (e.g., 91st percentile for PWS, and 72nd percentile for MWS) can be visualized in the VR/AR environment while simultaneously viewing the 3D models. The data processed is from a database that is growing and can be used to compare a patient aneurysms to a growing population.

FIG. 4 illustrates an example for leveraging AR to provide data-driven insights to a patient. In some examples, the process illustrated in FIG. 3 is leveraged to provide a framework to readily view the biomechanical wall stresses of cardiovascular diseases. Other biomechanical analysis can take upwards to 4 hours to manually segment medical imaging sets, extract the aneurysm geometry, and perform stress analysis (up to 24 hours). The framework disclosed herein may take less than 30 seconds to provide a heatmap of aneurysm wall stresses, allowing clinicians the ability to gain insights that typically require a biomechanics expert and expensive computational software. In some examples, a system to visualize the biomechanical wall stresses of cardiovascular disease in real-time is disclosed.

FIG. 5 illustrates an example AAA risk assessment visualization 500 with a chart presenting a comparison of a patient's condition versus a general population. In some examples, the visualization provides advanced analytics and population statistics for an expanding computational database to aid clinicians and/or patients during patient follow-up visits and/or telemedicine sessions.

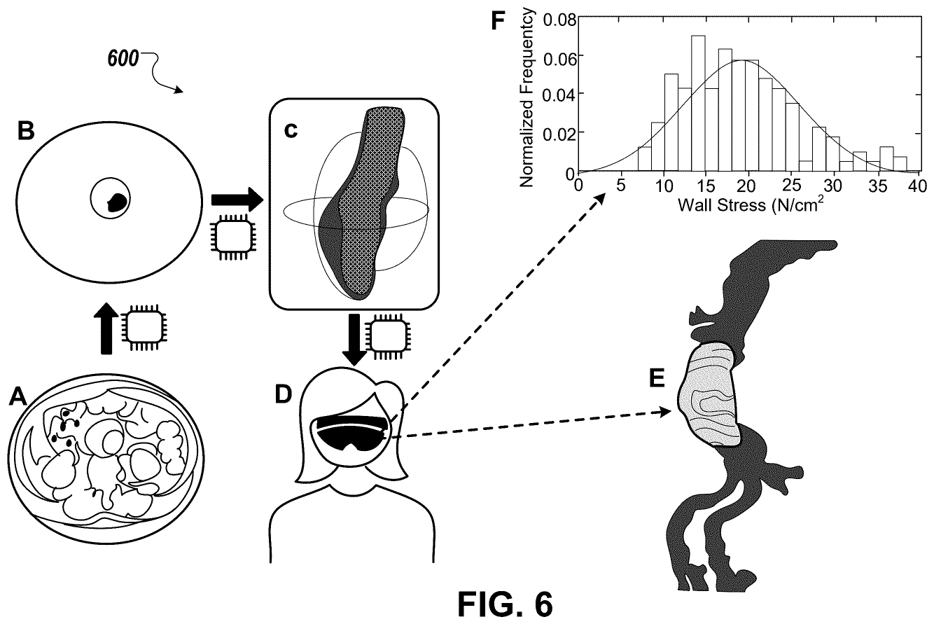

FIG. 6 illustrates an example process 600 that enables a user to view AAA risk assessment visualizations. The example shown includes steps for automatically segment regions of interest, project 3D objects overlayed on the original medical image set, and provide a heatmap of wall stresses with data insights to compare the disease state with the general population. The example process shown includes steps A, B, C, D, E, and F.

Step A shows an example of an axial slice of an anatomical scan for abdominal aortic aneurysm. At step B, image sets are input into a trained image classifier. A prepared dataset is input into a trained machine learning regression model to predict the wall stresses on the surface of the aneurysm. to automatically segment, resulting in a segmented region of interest (aneurysm wall and lumen). At step C, point clouds are converted and preprocessed. Step D shows an AR headset that is linked to a computational workstation allows the clinician to view the segmented medical image with stress analysis (shown at step E) and additional user controls based on gestures and/or speech commands allows for additional population statistics and data analytics to be displayed in real time to assess a patient's risk of rupture (shown at step F).

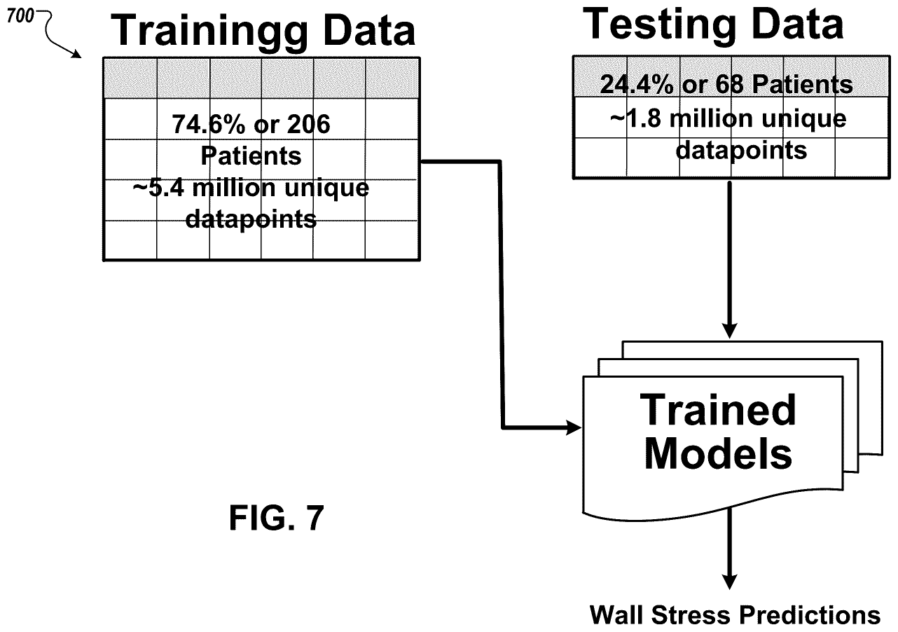

FIG. 7 is a schematic flow diagram illustrating an example process 700 for training models to make wall stress predictions. Input data is divided between training data and testing data (e.g., training data with annotations for the results being removed or hidden). Examples for training models to make wall stress predictions are disclosed herein.

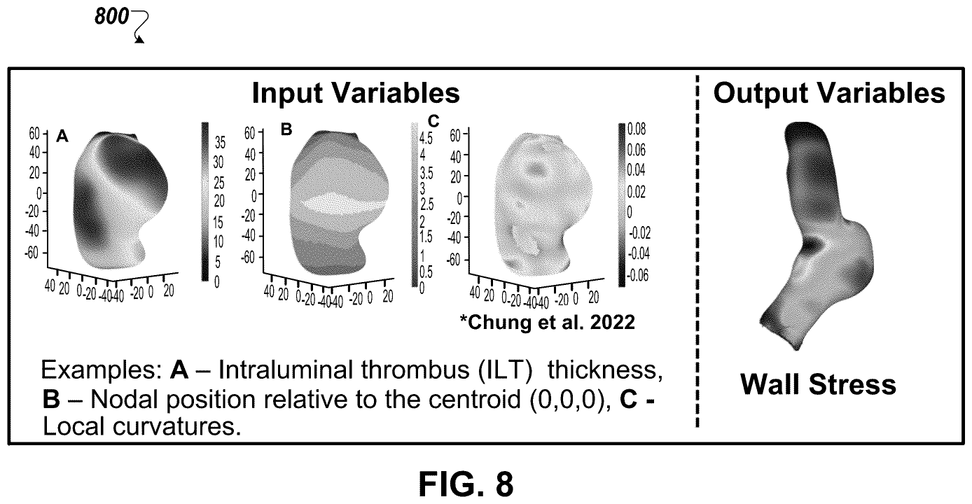

FIG. 8 illustrates example variables 800 for an example model used to predict wall stresses for an organ. The example shown includes input variables and an output variable. The input variables include intraluminal thrombus (ITL) thickness (a), nodal position relative to the centroid (0,0,0), and local curvatures (c). The output variable includes a 3D heatmap for predicted wall stress.

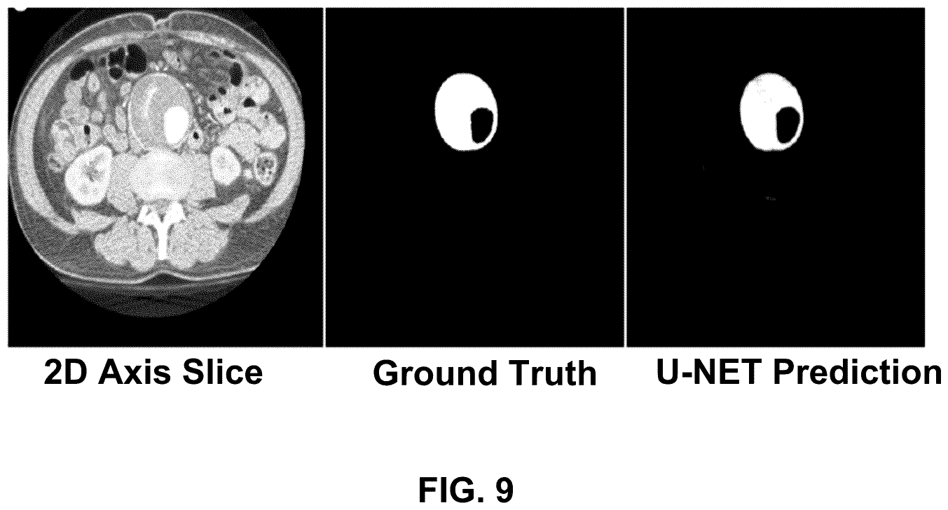

FIG. 9 illustrates example inputs and an output prediction for training a classifier to segment medical data. In some examples, a U-NET is trained from CT images that imaged abdominal aortic aneurysms. In some examples, each slice is manually segmented, labeled, and confirmed as an appropriate segmentation by a clinician. In some examples, the U-NET predicts image segmentation by piecing together sub-images.

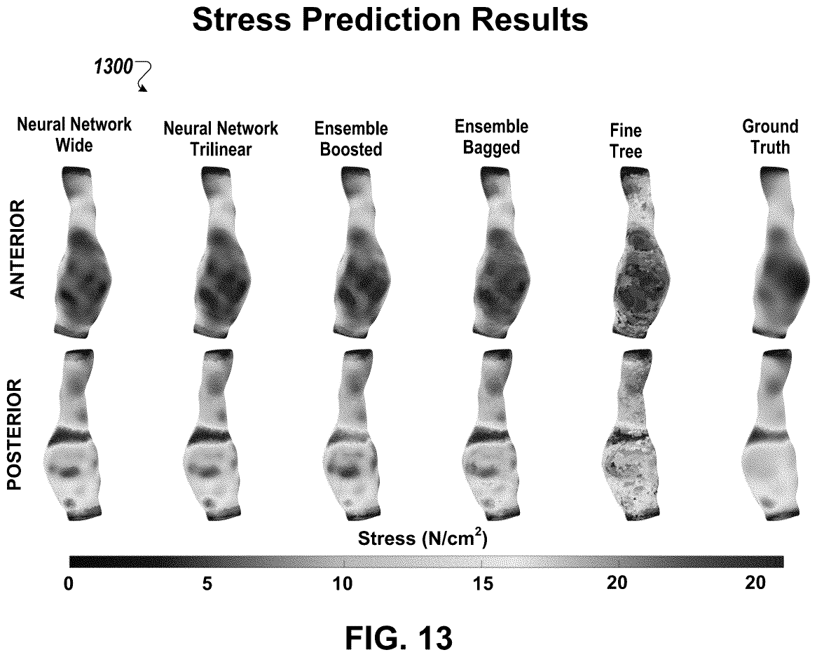

In some examples, a convolutional neural network (e.g., U-NET) image classifier trained using patient image sets and manually segmented ground truth images for abdominal aortic aneurysms. The U-NET predicts the aneurysm wall and lumen (where the blood flows) to capture the appropriate region of interest for 3D geometric surface reconstruction (FIG. 9). The 3D surface reconstructed models were pre-processed to prepare a dataset with computed wall stresses. In some examples, Machine Learning (ML) is used to train a ML regression model based on morphological indices to predict the wall stresses. FIG. 9 illustrates an example for training a classifier to segment medical data. Other techniques can be used including using other types of classifiers or machine learning technologies. For example, the machine learning technologies described in reference to FIG. 13.

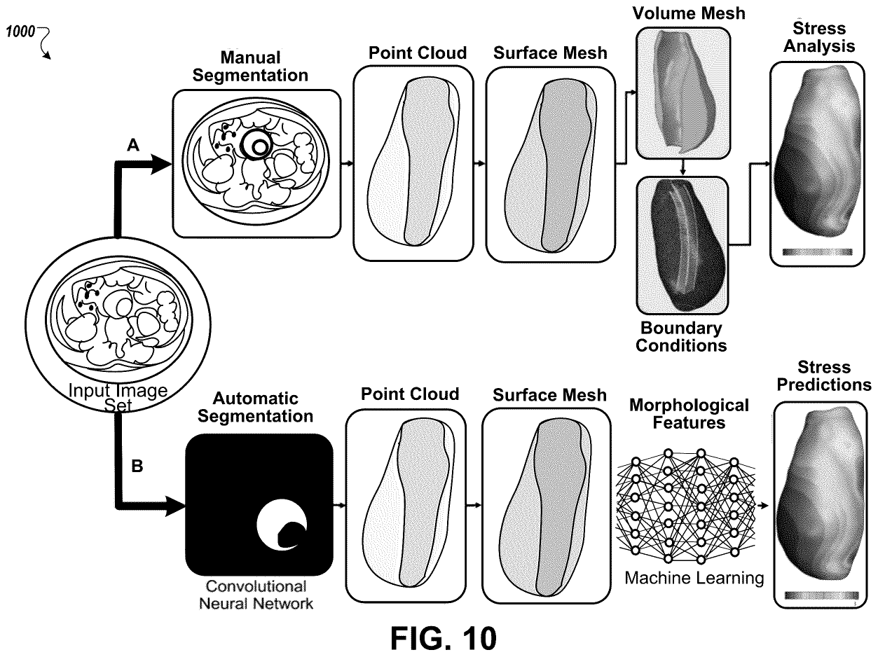

FIG. 10 illustrates an example process 1000 for predicting wall stresses. In some examples, the machine learning model is a convolutional neural network. FIG. 10 illustrates a pipeline to extract regions of interest from three-dimensional medical images, perform computational analysis, and visualize the stress heatmap of the 3D surface reconstructed geometry. In some examples, the results from the pipeline allow for the visualization of a heatmap of wall stresses acting on the aneurysm wall, providing insights for clinicians and experts to predict rupture or adverse patient outcomes. In some examples, artificial intelligence software toolkits and hardware (e.g., graphics processing units-GPUs).

The example shown includes a method for predicting wall stresses from medical images that leveraged convolutional neural networks (CNN) and machine learning (ML). An image classifier is trained using medical images from abdominal aortic aneurysms (AAA). In some examples, a clinical expert confirms the ground truth manual segmentations to input into the U-NET training pipeline. Training of the image classifier can be performed on a local workstation using (or alternatively in a cloud based system). The ML regression model to predict stresses is trained using preprocessed morphological indices and stress results from finite element analysis, and tree-based optimization pipeline.

The automatic segmentation may use a trained U-NET image classifier (a type of CNN) to automatically identify the regions of interest in a medical image stack to define a set of points, data-processing, and the eventual prediction of wall stresses using a trained ML regression model. The AI framework may take less than 30 seconds of total computational time. Accordingly, the AI framework to predict stresses on medical images provides the basis for real-time biomechanics-based visualization of cardiovascular diseases in medical images.

In the example shown, includes a pipeline for manually extracting regions of interest using manual segmentation, converting a region of interest into a point cloud, and performing meshing and stress analysis. Also shown is a pipeline for automatically extracting regions of interest from medical images using an artificial intelligence framework and predicting wall stresses using a trained machine learning model.

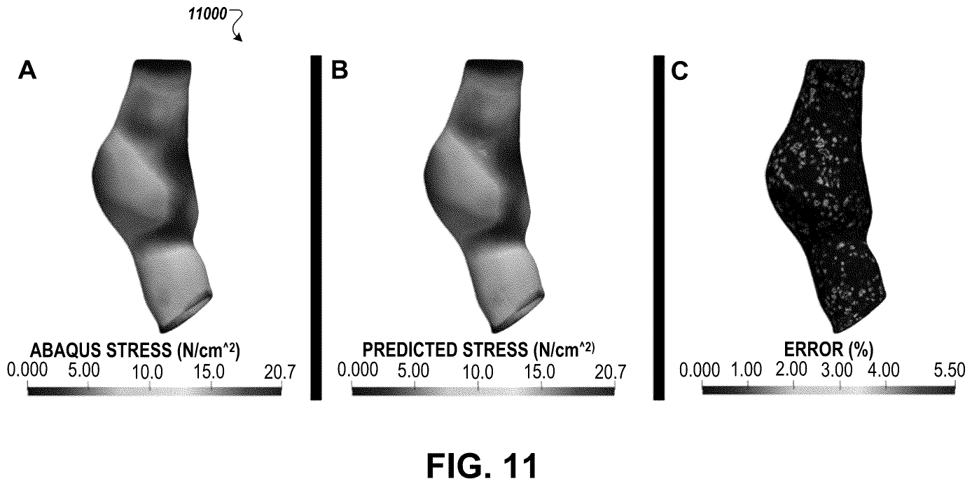

FIG. 11 illustrates wall stress heatmap using computational simulations (A), predicted wall stresses based on the ML prediction model (B), and percent errors at each node of the stress differences (C). The result of wall stress predictions using the same input 3D surface reconstructed geometry. A comparison can be made between A and B, with the percent error plotted for each node in C.

A database of medical images of abdominal aortic, ascending aortic, and brain aneurysms can be housed on local servers. For example, including a dataset of 700 abdominal aneurysms processed using stress analysis that is used to train a larger ML regression model. The methods outlined in herein can further be implemented on ascending and cerebral aneurysms.

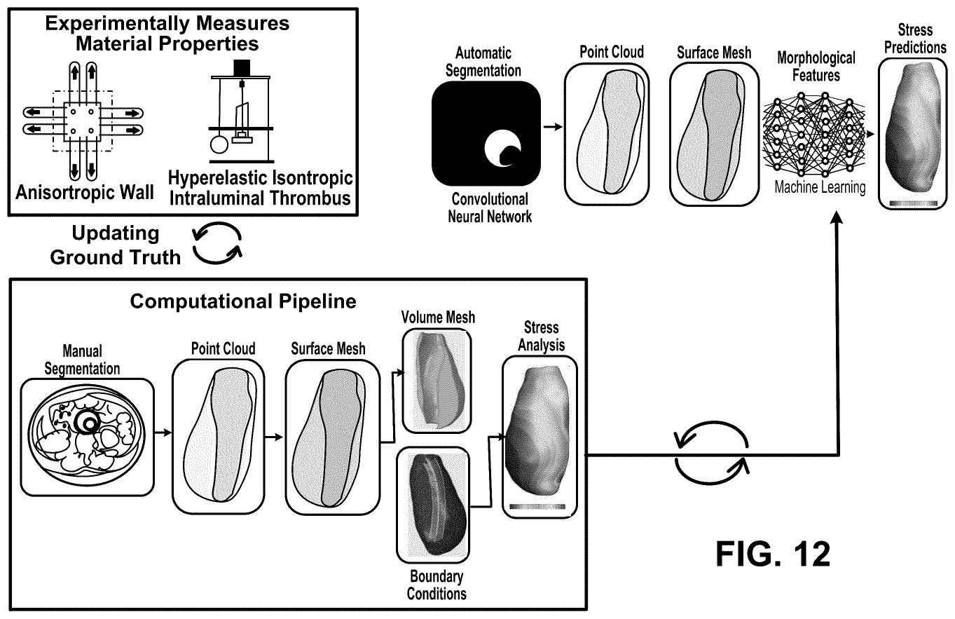

FIG. 12 illustrates an example process 1200 for implementing the artificial intelligence pipeline with continuous and incremental learning. In some examples, after a suitable sample size for training is found, an expedited feedback process to re-run stress analysis on newly developed material models from experimental measurements is implemented to allow for continual updates to the ML stress prediction models.

FIG. 13 illustrates example stress prediction results 1300 different machine learning models (ensembled trees (e.g., bagged and boosted), tree models (e.g., fine and coarse), and neural networks (e.g., wide, trilinear)). The figure shows the ground truth on the first column, and the results of the various methods showing the anterior and posterior views. In this example, the wide neural network performed the best among the trained models. The wall stress map shows elevated stress banding on the posterior section of the aneurysm.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory program carrier for execution by, or to control the operation of, data processing apparatus. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus. The computer storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Computers suitable for the execution of a computer program include, by way of example, general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube), LCD (liquid crystal display) monitor, or OLED (organic light-emitting diode) for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the user device, which acts as a client. Data generated at the user device, e.g., a result of the user interaction, can be received from the user device at the server.

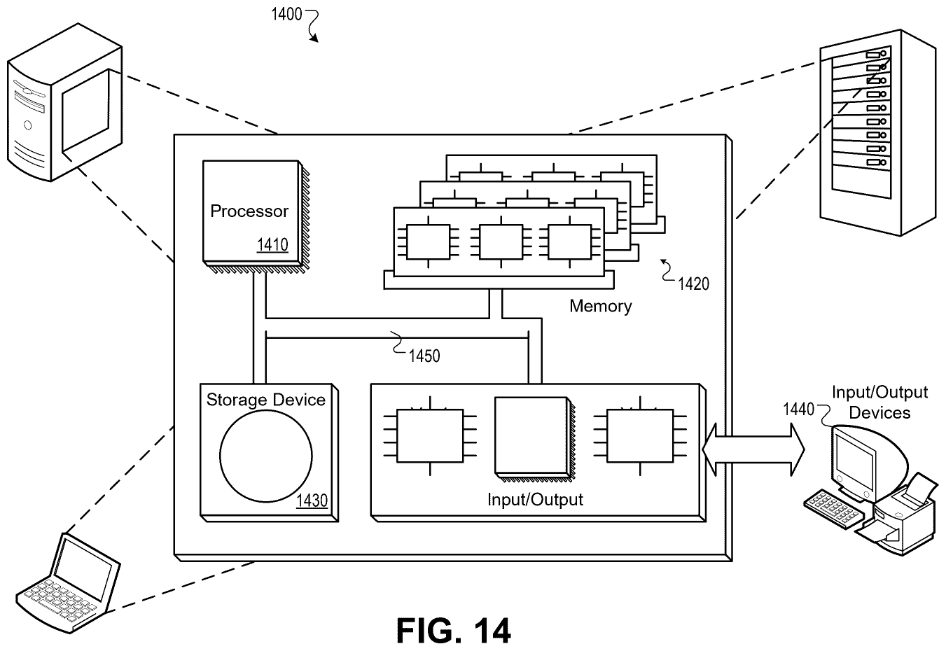

An example of one such type of computer is shown in FIG. 14, which shows a schematic diagram of a generic computer system 1400. The system can be used for the operations described in association with any of the computer-implemented methods described previously, according to one implementation. The system 1400 includes a processor 1410, a memory 1420, a storage device 1430, and an input/output device 1440. Each of the components 1410, 1420, 1430, and 1440 are interconnected using a system bus 1450. The processor 1410 is capable of processing instructions for execution within the system 1400. In one implementation, the processor 1410 is a single-threaded processor. In another implementation, the processor 1410 is a multi-threaded processor. The processor 1410 is capable of processing instructions stored in the memory 1420 or on the storage device 1430 to display graphical information for a user interface on the input/output device 1440.

The memory 1420 stores information within the system 1400. In one implementation, the memory 1420 is a computer-readable medium. In one implementation, the memory 1420 is a volatile memory unit. In another implementation, the memory 1420 is a non-volatile memory unit.

The storage device 1430 is capable of providing mass storage for the system 1400. In one implementation, the storage device 1430 is a computer-readable medium. In various different implementations, the storage device 1430 may be a floppy disk device, a hard disk device, an optical disk device, or a tape device.

The input/output device 1440 provides input/output operations for the system 1400. In one implementation, the input/output device 1440 includes a keyboard and/or pointing device. In another implementation, the input/output device 1440 includes a display unit for displaying graphical user interfaces.

Other embodiments and applications not specifically described herein are also within the scope of the following claims. Elements of different implementations described herein may be combined to form other embodiments not specifically set forth above. Elements may be left out of the structures described herein without adversely affecting their operation. Furthermore, various separate elements may be combined into one or more individual elements to perform the functions described herein. The following are numbered embodiments intended to further illustrate, but not limit, the scope of the invention.

Embodiment 1 is a method, comprising: obtaining, by a system of one or more computers, medical image data of a person, the medical image data comprising one or more images that each depict at least a portion of a vascular disease; generating, by the system and using the medical image data, a three-dimensional (3D) model of the vascular disease, the 3D model defining an outer wall of the vascular diseases; analyzing, by the system, the 3D model of the vascular disease to determine respective values for at least one morphological feature of the vascular disease and at least one biomechanical feature of the vascular disease; using the respective values for the at least one morphological feature of the vascular disease and the at least one biomechanical feature of the vascular disease to predict one or more outcomes related to the vascular disease; and outputting a visualization of a risk assessment for the vascular disease based on the predicted one or more outcomes related to the vascular disease, wherein the visualization is configured to be rendered in at least one of augmented reality (AR) or virtual reality (VR).

Embodiment 2 is the method of embodiment 1, wherein the visualization includes a 3D rendering of the 3D model of the vascular disease overlayed with a heat map corresponding to the at least one morphological feature.

Embodiment 3 is the method of any one of embodiments 1-2, wherein the visualization includes a graph presenting a comparison of the at least one morphological feature to the at least one morphological feature for a general population.

Embodiment 4 is the method of any one of embodiments 1-3, wherein the visualization includes: a 3D rendering of the 3D model of the vascular disease overlayed with a heat map corresponding to the at least one morphological feature; and a graph presenting a comparison of the at least one morphological feature to the at least one morphological feature for a general population.

Embodiment 5 is the method of any one of embodiments 1-4, wherein the visualization includes an animation presenting a change of the at least one morphological feature over a period of time based on the predicted one or more outcomes related to the vascular disease.

Embodiment 6 is the method of embodiment 5, wherein the animation presenting the change over the period of time includes an animation for a change in surface geometry.

Embodiment 7 is the method of any one of embodiments 5-6, wherein the animation presenting the change over the period of time includes an animation for a change in wall stresses represented by a heatmap.

Embodiment 8 is the method of any one of embodiments 1-7, the method further comprising: generating a timeline of key events based on the predicted one or more outcomes related to the vascular disease; and wherein the visualization includes 3D visualizations for each of the key events.

Embodiment 9 is the method of any one of embodiments 1-8, the method further comprising: calculating metrics related to the at least one morphological feature; and selecting one or more of the metrics related to the at least one morphological feature to include in the visualization.

Embodiment 10 is the method of embodiment 9, wherein an AR/VR viewer interface presents the selected one or more metrics on a virtual 3D object.

Embodiment 11 is the method of embodiment 10, wherein the virtual 3D object is a cube.

Embodiment 12 is the method of any one of embodiments 1-11, wherein the visualization is configured to be viewed by a medical provider to assess a condition of the vascular disease.

Embodiment 13 is the method of any one of embodiments 1-12, wherein the visualization is configured to be viewed by a patient and a medical provider as part of a telemedicine application.

Embodiment 14 is the method of embodiment 13, wherein the medical provider and the patient are viewing a same instance of the visualization on separate AR/VR devices.

Embodiment 15 is the method of any one of embodiments 1-14, wherein the vascular disease is an aneurysm.

Embodiment 16 is the method of embodiment 15, wherein the aneurysm is an abdominal aortic aneurysm.

Embodiment 17 is the method of embodiment 15, wherein the aneurysm is a cerebral aneurysm.

Embodiment 18 is one or more non-transitory computer-readable media having instructions stored thereon that, when executed by one or more processors, cause performance of any of the methods of embodiments 1-17.

Embodiment 19 is a system comprising: one or more computers; and one or more computer-readable media having instructions stored thereon that, when executed by the one or more computers, cause performance of any of the methods of embodiments 1-17.

Embodiment 20 is a method comprising: obtaining, by a system of one or more computers, medical image data of a patient, the medical image data comprising one or more images that each depict at least a portion of an aneurysm; generating a 3D point cloud by processing the medical image data; converting the 3D point cloud to a 3D surface comprising a plurality of nodes defined by coordinates; predicting wall stresses for each of the plurality of nodes using a machine learning model; generating a visualization of a risk assessment for the aneurysm by combining the 3D surface with a heatmap corresponding to the predicted wall stresses at each of the plurality of nodes; and providing the visualization of the risk assessment to at least one of an augmented reality device or virtual reality device to display the visualization.

Embodiment 21 is the method of embodiment 20, wherein the medical image data is processed using a classifier to identify a segment of medical image data that includes a region of interest.

Embodiment 22 is the method of any one of embodiments 20-21, wherein inputs to the machine learning model include aneurysm wall coordinates, intra-luminal thrombus (ILT) thickness, nodal neighbors, principal curvatures, and distance to centroid.

Embodiment 23 is the method of any one of embodiments 20-22, the method further comprising: generating two-dimensional (2D) graphics to compare peak wall stresses and mean wall stresses of the aneurysm for the patient to peak wall stresses and mean wall stresses of aneurysm for a population of patients; wherein the visualization includes the 2D graphics.

Embodiment 24 is one or more non-transitory computer-readable media having instructions stored thereon that, when executed by one or more processors, cause performance of any of the methods of embodiments 20-23.

Embodiment 25 is a system comprising: one or more computers; and one or more computer-readable media having instructions stored thereon that, when executed by the one or more computers, cause performance of any of the methods of embodiments 20-23.

While this specification contains many specific implementation details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

1. A method, comprising:
obtaining, by a system of one or more computers, medical image data of a person, the medical image data comprising one or more images that each depict at least a portion of a vascular disease;
generating, by the system and using the medical image data, a three-dimensional (3D) model of the vascular disease, the 3D model defining an outer wall of the vascular diseases;
analyzing, by the system, the 3D model of the vascular disease to determine respective values for at least one morphological feature of the vascular disease and at least one biomechanical feature of the vascular disease;
using the respective values for the at least one morphological feature of the vascular disease and the at least one biomechanical feature of the vascular disease to predict one or more outcomes related to the vascular disease; and
outputting a visualization of a risk assessment for the vascular disease based on the predicted one or more outcomes related to the vascular disease, wherein the visualization is configured to be rendered in at least one of augmented reality (AR) or virtual reality (VR).

2. The method of claim 1, wherein the visualization includes a 3D rendering of the 3D model of the vascular disease overlayed with a heat map corresponding to the at least one morphological feature.

3. The method of claim 1, wherein the visualization includes a graph presenting a comparison of the at least one morphological feature to the at least one morphological feature for a general population.

4. The method of claim 1, wherein the visualization includes:
a 3D rendering of the 3D model of the vascular disease overlayed with a heat map corresponding to the at least one morphological feature; and
a graph presenting a comparison of the at least one morphological feature to the at least one morphological feature for a general population.

5. The method of claim 1, wherein the visualization includes an animation presenting a change of the at least one morphological feature over a period of time based on the predicted one or more outcomes related to the vascular disease.

6. The method of claim 5, wherein the animation presenting the change over the period of time includes an animation for a change in surface geometry.

7. The method of claim 5, wherein the animation presenting the change over the period of time includes an animation for a change in wall stresses represented by a heatmap.

8. The method of claim 1, the method further comprising:
generating a timeline of key events based on the predicted one or more outcomes related to the vascular disease; and
wherein the visualization includes 3D visualizations for each of the key events.

9. The method of claim 1, the method further comprising:
calculating metrics related to the at least one morphological feature; and
selecting one or more of the metrics related to the at least one morphological feature to include in the visualization.

10. The method of claim 9, wherein an AR/VR viewer interface presents the selected one or more of the metrics on a virtual 3D object.

11. The method of claim 10, wherein the virtual 3D object is a cube.

12. The method of claim 1, wherein the visualization is configured to be viewed by a medical provider to assess a condition of the vascular disease.

13. The method of claim 1, wherein the visualization is configured to be viewed by a patient and a medical provider as part of a telemedicine application.

14. The method of claim 13, wherein the medical provider and the patient are viewing a same instance of the visualization on separate AR/VR devices.

15. The method of claim 1, wherein the vascular disease is an aneurysm.

16. The method of claim 15, wherein the aneurysm is an abdominal aortic aneurysm.

17. The method of claim 15, wherein the aneurysm is a cerebral aneurysm.

18. A system comprising:
one or more computers; and
one or more computer-readable media having instructions stored thereon that, when executed by the one or more computers, cause the system to:
obtain medical image data, the medical image data comprising one or more images that each depict at least a portion of a vascular disease;
generate, using the medical image data, a three-dimensional (3D) model of the vascular disease, the 3D model defining an outer wall of the vascular diseases;
analyze the 3D model of the vascular disease to determine respective values for at least one morphological feature of the vascular disease and at least one biomechanical feature of the vascular disease;
use the respective values for the at least one morphological feature of the vascular disease and the at least one biomechanical feature of the vascular disease to predict one or more outcomes related to the vascular disease; and
output a visualization of a risk assessment for the vascular disease based on the predicted one or more outcomes related to the vascular disease, wherein the visualization is configured to be rendered in at least one of augmented reality (AR) or virtual reality (VR).

19. The system of claim 18, wherein the medical image data is processed using a classifier to identify a segment of the medical image data that includes a region of interest.

20. The system of claim 18, wherein the vascular disease is an aneurysm and the system is further cause to:
generate two-dimensional (2D) graphics to compare peak wall stresses and mean wall stresses of the aneurysm to peak wall stresses and mean wall stresses of aneurysm for a population of patients; and
wherein the visualization includes the 2D graphics.
